(19) United States
(12) Patent Application Publication
(10) Pub. No.: US 2013/0079620 A1
(43) Pub. Date: Mar. 28, 2013

(54) METHOD FOR PROCESSING AN ENDOSCOPY IMAGE

(71) Applicants: Rainer Kuth, Hochstadt (DE); Markus Neurath, Erlangen (DE)

(72) Inventors: Rainer Kuth, Hochstadt (DE); Markus Neurath, Erlangen (DE)

(73) Assignee: Siemens AG, Munchen (DE)

(21) Appl. No.: 13/577,035

(22) PCT Filed: Feb. 2, 2011
(86) PCT No.: PCT/EP11/51425

§ 371 Date: Dec. 13, 2012

(30) Foreign Application Priority Data
Feb. 3, 2010 (DE) 10 2010 006 741.5

(51) Int. Cl. A61B 1/04; G06T 7/00

(52) U.S. Cl. CPC A61B 1/04; G06T 7/0012
USPC 600/407; 382/128

(57) ABSTRACT

In a method for processing a generated digital endoscopy image of a patient, those pixels that depict a body region of a patient are determined as image points in the endoscopy image, in a processor. In the processor, a color value is derived for image point using the value of the associated pixel, and an area portion associated with each image point is derived for the color value. A color area is derived for each color value, as the sum of all area portions of the image points having that color value. An evaluation measure of the endoscopy image is then implemented in the processor using the respective color areas.

### BACKGROUND OF THE INVENTION

1. Field of the Invention

The invention concerns a method to process a digital endoscopy image of a patient that is generated.

2. Description of the Prior Art

The endoscopic examination or treatment of patients is a widespread medical measure. In endoscopic interventions, endoscopy images (normally digital) of the patient are generated. The images are displayed on a monitor to the endoscopist. An endoscopy is frequently performed in the gastrointestinal tract of a patient, for example. The endoscopist conducts a visual observation and assessment of the images by observing, for example, color distribution and texture of the mucous membrane or surface of the gastrointestinal tract that is imaged in the endoscopy image. From this, the endoscopist obtains findings and combines these with his personal experiences and optional additional information about the examined patient. The quality of this procedure therefore decisively depends on the qualification of the endoscopist.

In various patients, follow-up examinations—i.e. additional endoscopies at later points in time—are always conducted again in order to track the progress of an illness in the patient, for example. In the known procedure, the respective current examination result—i.e. the currently acquired endoscopy images—can be only compared qualitatively with earlier examinations. For example, for this purpose the endoscopist can draw upon archived endoscopy images or proceed according to his own memories.

The goal of an endoscopy is, for example, to detect or to diagnose an inflammation in the gastrointestinal tract of a patient. The endoscopist makes a judgment as to whether the respective examined patient has inflammations or not purely via the observation of the endoscopy image—i.e. his personal color perception—using the shown patient surface. The endoscopist is hereby reliant upon his subjective perception, as well as on the color differences that are perceptible in the first place to the human eye.

The visual perception of humans takes place via receptors that are located on the retina. These receptors are rods for the light/dark contrast, cones for color perception. Cones are present in three forms that have their sensitivity maximum in the spectral ranges “red”, “green” and “blue”. Colors can therefore be represented as a three-dimensional property due to the three types of color receptors in humans. Each combination of excitations of the three cone types by light radiation that strikes the retina produces a specific color impression. Black (no excitation at all), neutral grey and white (i.e. full and simultaneous excitation of all three cone types) are thus also likewise colors that are classified as achromatic colors.

The one-dimensional representation of the spectral colors as it occurs via refraction into rainbows or after a prism (represented as a color wheel of the chromatic colors) includes only a few color perceptions. Visible radiation is an electromagnetic radiation in the wavelength range from 380 nm to 780 nm. A color perceptible to humans thus can be defined by three parameters. If the absolute brightness is now eliminated, two parameters remain, meaning that all colors can be depicted in a 2D space together with a one-dimensional luminosity.

The aforementioned color perception is a subjective process that proceeds differently for every person. Since human color perception or, respectively, image perception of the endoscopy image is decisive for the endoscopy that is described above, evidence-based medicine is not conducted here. The latter requires quantitative measurement values during the course of an illness, wherein the measurement values must be determinable independent of the examiner. Such a thing is also desirable for endoscopy.

A computer-assisted diagnosis method (CAD) is known from US 2007/0135715 A1. A number of endoscopy images which are generated by an endoscopy capsule are assessed there automatically for hemorrhage locations in the patient.

A method for analytical detection of a macular degeneration is known from AT 503 741 A2. Images of the background of the eye of a patient which are generated via fluorescence angiography and similar imaging methods are thereby classified via a four-stage method.

### SUMMARY OF THE INVENTION

It is the object of the present invention to specify an assistive method that allows an improved endoscopy.

In other words, the object is to specify a technique or a method with which quantitative examination results can be generated in endoscopy. The invention is based on the idea to introduce for this purpose a method to process a digital image of a patient that is generated.

The object is achieved by a method according to the invention that is based on a digital endoscopy image of the patient that was previously generated, which digital endoscopy image is processed according to the method. In the endoscopy image various pixels are determined as image points, wherein the image points are additionally processed later in the method. At least a portion of those pixels that depict a region of the body of the patient are selected as image points. In other words, only image contents of the endoscopy image which actually represent an image of the patient are thus processed in the method. Pixels that (for example) depict an instrument situated in the field of view of the endoscope are not taken into account.

A color value is now determined for each image point. This occurs using the value of the pixel associated with this image point. Moreover, an area proportion associated with the image point is determined for each image point. As is explained further below, the area proportion can hereby relate to the area of the endoscopy image or the real area of the patient or, secondly, of the portion of the patient that is depicted in the image.

If all desired or all available image points in the endoscopy image are defined and their corresponding color values and area proportions are determined, a color area is subsequently determined for each determined color value: the sum of all area proportions whose image points have the appertaining color value is hereby calculated. In other words, for each color rotation determined in the endoscopy image in the form of a specific color value, it is determined what area proportion of the endoscopy image or of the patient has this one color value. The corresponding measure of this is the respective color area associated with the color value.

Finally, the evaluation measure for the endoscopy image is determined using the determined color areas. Again, different variants described further below are also provided for this purpose.

In other words, according to the invention a form of color map in which the respective area proportions in the image or on the patient which have a defined color or, respectively, are associated with a color group are determined is thus created for an endoscopy image of the patient that is acquired. The evaluation measure is determined from the corresponding color areas according to a specific algorithm or, respectively, specific rules or a specific method. Each endoscopy image is therefore evaluated in a reproducible and quantitative manner and delivers a measurement value that enables evidence-based medicine within the scope of endoscopy. Subjective color assessments by the endoscopist are eliminated. The evaluation measure is an objectively reproducible, quantitative measurement.

Since the method operates on the pixel values of the digital endoscopy image, the evaluation measure simply does not run up against the aforementioned limits of the human color perception; rather, it is only dependent on the technical differentiation capability of the generation of the endoscopy image or, respectively, on the corresponding bit resolution in the generation of the endoscopy image. Within the scope of the method, alternative endoscopy techniques can also be used which deliver information in the endoscopy image that goes beyond the resolution capability of the human eye but can be used by the method. Diagnoses can therefore also be improved beyond the characteristics of human color perception if information in the endoscopy image that is not perceptible to the human eye enters into the evaluation measure.

As was mentioned above, multiple variants exist for the determination of the area proportion.

In a first embodiment of the method, a value correlated with the area of the pixel in the endoscopy image that is associated with this image point is determined as an area proportion of said image point. The determined area proportions are thus oriented towards the image area of the endoscopy image. Such a determination of an area proportion is particularly simple. For example, the actual area of a pixel in an image is measured as an area proportion.

In an alternative embodiment, a value correlated with the area of the patient that is depicted at the image point is determined as an area proportion of said image point. Here the area proportion is oriented towards the actual surface of the body of the patient which is depicted in the image. For example, here the viewing angle of the endoscope towards the patient surface is to be taken into account in order to determine the actual patient area that is depicted at the image point. In this method variant, the area proportion is better related to the actual patient and delivers a measurement value which is oriented towards the actual patient surface.

The area proportion is always a value that is correlated only with the area, thus does not need to represent the absolute assessed area; rather, it can merely be in relation to this, for example. Given orientation towards the image area for an image point, the area proportion can always be counted as “1”, for example.

As was mentioned above, variants also exist for the determination of the evaluation measure:

In a first embodiment, a total area entering into the evaluation measure is determined as a reference with regard to the respective color areas. The color areas can thus be related to the reference and a percentile or, respectively, ratio value can enter into the determination of the evaluation measure. Various possibilities which normally are used in the method together with the aforementioned possibilities for the determination of the area proportions result again for the total area.

In a first embodiment, the image area of the endoscopy image is determined as a total area. In combination with the aforementioned determination of the area proportions using the areas of the pixels in the endoscopy image, total area and area proportions are thus oriented towards the area of the endoscopy image. Alternatively, the sum of all area proportions can also be determined as a total area if not all image contents depict the patient, for example.

In an alternative embodiment, the area of the patient that is depicted in the endoscopy image is determined as a total area. This variant is normally combined with the aforementioned variant in which the area proportions are also oriented towards the actual patient surface.

In a preferred embodiment of the method, the evaluation measure is determined as a quotient of color area and total area of one, multiple or all color areas. In other words, the evaluation measure then yields conclusions about with which proportions specific colors of the patient surface are present in the endoscopy image.

In another embodiment, a histogram of the color areas across the color values is determined as an evaluation measure. Here the color areas are thus not linked with a total area; rather, only their respective proportions are presented in the form of a histogram.

Various possibilities also exist for the determination of the color value of an image point:

For example, the corresponding pixel value of the associated pixel in the endoscopy image can be selected as a color value.

In a further embodiment, however, the color value is selected so that it is correlated with a pathological property of the patient. For example, only two color values are assigned for the assessment of an endoscopy image, namely one that corresponds to an inflamed body region of a patient and one that corresponds to a body region of a patient that is not inflamed. Only one of the two color values is respectively associated with the image points. For this the pixel values of the corresponding image pixels are evaluated and classified in the corresponding color values. Each color value therefore corresponds to an entire value range of pixel values of the image pixels.

In a preferred embodiment of the method, value ranges of pixel values are therefore used as color values.

Such color values can be understood as color clusters, wherein the properties of the respective cluster can be selected.

In a particularly preferred variant of this embodiment, the values ranges are selected using a standard color system. For example, finitely large color ranges—thus color ranges of pixel values—are connected to clusters according to the RAL color system (RAL Deutsches Institut fur Gutesicherung and Kennzeichnung e.V.); for example, all colors between RAL-3014 (antique pink) and RAL-3033 (pearl pink) are combined into a cluster which then corresponds to a body tissue of the patient that is not inflamed.

Value ranges—thus the association or, respectively, classification of which pixel values are associated with which color values—can be predeterminable as a standard from a central location or, respectively, can be loaded into the respective endoscopy system, for example. Endoscopy images that are processed with such a standard association are then comparable among one another. For this purpose it is merely necessary that the endoscopy images have comparable color reproduction, thus are color-calibrated, for example. The values ranges can be loaded from a standard medium such as a diskette, for example. It is also possible to load the standard values from the Internet, thus for example from a central location such as an Internet server.

The pixel values that establish the color of a pixel are normally value groups made up of multiple color channels. For example, a pixel thus comprises a red value, green value and blue value, respectively in the form of a digital value, wherein each value corresponds to a color channel. In a preferred embodiment of the method, the color value is determined using a mask applied to the color channels. The classification of pixels into various color values then takes place via a mask comparison and is to be implemented particularly simply and quickly. As mentioned above, the method opens up the possibility to also provide the pixels of the endoscopy images with more color channels than the human eye could resolve. Classifications—and thus the creation of the evaluation measure—can thus exceed the capability of solely an optical consideration by a human observer.

In an alternative method variant, a luminosity-normalized 2D value is selected as a color value. The decomposition of colors into a 2D color value and a luminosity was explained above. By eliminating the luminosity, the classification then actually takes place only according to the color tone and not according to its saturation in the form of the luminosity.

In one advantageous embodiment of the method, image points are respectively determined as groups of pixels in the endoscopy image. In other words, the resolution of the endoscopy image is thus reduced for the method steps (and ultimately for the determination of the evaluation measure); for example a 3×3 field of pixels in the endoscopy image is respectively averaged or, respectively, defined as a single image point.

In a preferred embodiment of the method, the evaluation measure is determined for multiple endoscopy images. Within the scope of an endoscopy, not just a single endoscopy image but rather an image series is normally created. The evaluation measure can then be determined for each image (or at least multiple images) of the image series. For example, image points can hereby be disregarded that were already depicted in a prior endoscopy image.

In a preferred embodiment, the method can also be applied to an endoscopy image which, as a sum image, is composed from individual images. For example, the image series acquired in the course of an endoscopy can be assembled (within the scope of a “stitching”) into a single image (enlarged relative to the individual images) of the entire endoscoped patient region, and the method can be applied to the assembled image.

It is then taken into account whether specific segments of the patient are depicted twice in multiple endoscopy images. The double or multiple depiction of the same points of the patient surface can correspondingly affect the determination of the color values (averaging over various pixels, i.e. various viewing angles of one and the same point of the patient) or can also affect the calculation of the patient area for the area proportion or the total area, for example.

In a further preferred embodiment, a 3D data set is used as an endoscopy image. The image can be designed as a virtual 3D image surface, particularly given assembly or, respectively, stitching of multiple endoscopy images of a three-dimensional hollow organ. The method can also be applied to such a data set.

Naturally, a calibration of the method normally takes place (for example using a RAL color map) in order to respectively obtain results that are comparable between different patients or different endoscopy images.

### BRIEF DESCRIPTION OF THE DRAWING

The single figure schematically illustrates implementation of an endoscopy procedure in accordance with the present invention.

### DESCRIPTION OF THE PREFERRED EMBODIMENTS

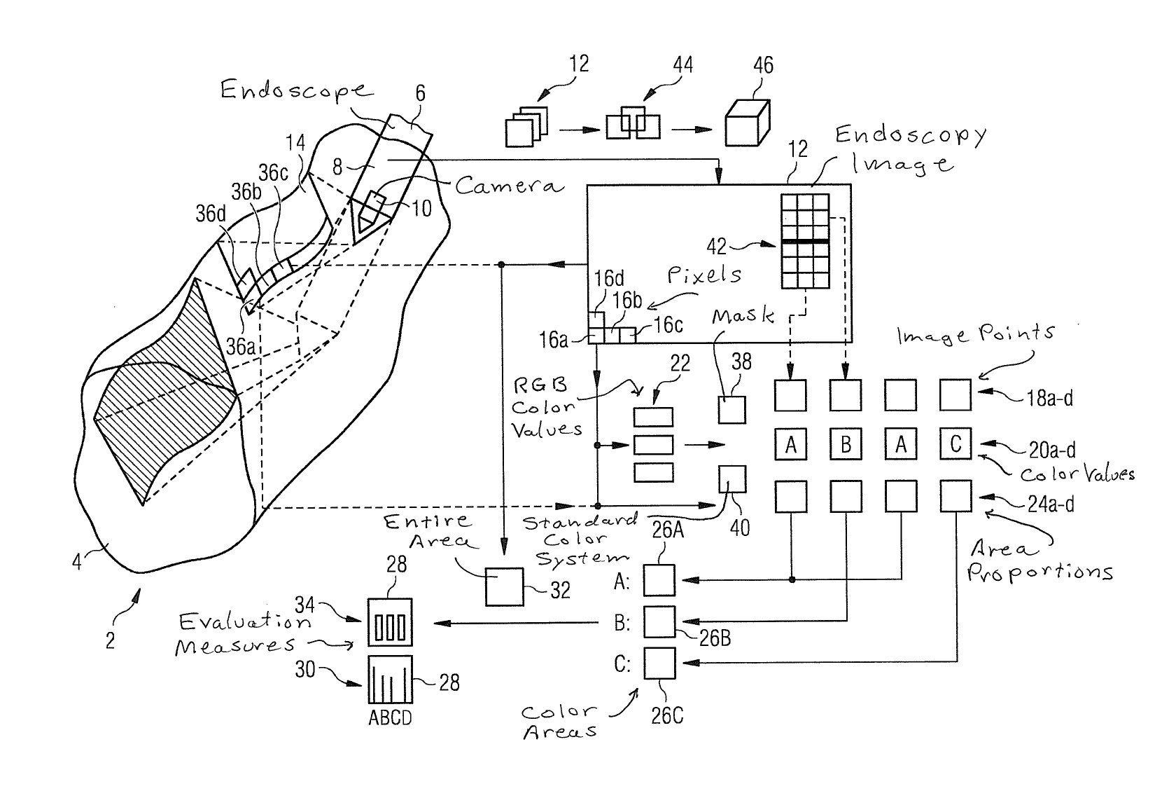

The figure shows a section from a patient 2, namely a segment from his esophagus 4. An endoscopy is presently conducted on the patient 2, which is why an endoscope 6 is introduced into the esophagus 4. A camera 10 that delivers an endoscopy image 12 of the inside of the patient is mounted at the end 8 of the endoscope. The viewing angle of the camera 10 as well as the region 14 of the inner wall of the esophagus 4 that are depicted in the endoscopy image 12 are represented with dashed lines in FIG. 1. The endoscopy image 12 is made up of individual pixels, of which the pixels 16a-d are shown as examples.

The endoscopy image 12 is now processed according to the method according to the invention: for each pixel 16a-d a check is initially made as to whether it depicts a body region of the patient 2. In the example this is satisfied for all pixels. Each pixel 16a-d therefore represents an image point 18a-d that is to be examined further. An associated color value 20a-d is now determined for each image point 18a-d. For each pixel 16a-d, its respective color information is evaluated for this.

In a first embodiment, the color information of each pixel 16a-d is respectively a triplet of values, namely an RGB value 22 which respectively reflects the red, green and blue proportion of the color of the pixel 16a-d. The image points 18a and c respectively have the value A as an RGB value 22, the image point 18b has the value B and the image point 18d has the value C. These yield the values of the color values 20a-d. The value A hereby corresponds to a dark red, the value B corresponds to a medium red and the value C corresponds to a light red.

An area proportion 24a-d is now determined for each image point 18a-d. In a first exemplary embodiment, this area proportion 24a-d is respectively oriented towards the area of 0.01 mm² of the respective pixels 16a-c in the endoscopy image 12. A pixel is associated with a relative area proportion of “1”.

This process is now repeated for all 320×200 pixels of the endoscopy image 12. In the example another fourth value D is hereby found as a color value (a dark grey).

For each found value A-D of the color values 20a-d, the associated color area 26A-D of the respective image points 18a-d having the corresponding value A-D is now determined via summation. In the example with pixels 16a-d of equally large area, a color area 26A thus results for the value A of “2” that is twice as large as the respective color areas 26B and C with “1”. This process is now also implemented for all 320×200 pixels of the endoscopy image 12.

In a last step, an evaluation measure 28 is determined from the color areas 26A-D. In a first embodiment, this represents a histogram 30 of the respective color areas 26A-D over the color values A-D (for the entire image now, for example).

In an alternative embodiment, the entire area 32 of the endoscopy image 12 is additionally computed. Given 320×200 pixels, this yields a value of “64000”. As an alternative evaluation measure 28, here the respective ratios 34 of the respective color areas 26A-D to the area 32 are calculated.

The figure also shows the region 14 in the esophagus 4 of the patient 2 which is depicted in the endoscopy image 12 as well as imaging regions 36a-d of the patient 2 which are depicted in the respective pixels 16a-d. In an alternative embodiment, the area proportions 24a-d are determined correlated with the areas of the imaging regions 36a-d and the area 32 is determined correlated with the area of the region 14. The correspondingly determined areas or, respectively, area proportion then better reflect(s) the real area of the patient that is imaged, and not only the image area in the endoscopy image 12. For example, an area proportion 24a of “1.1” results here since the imaging region 36a lies at an angle relative to the viewing angle of the camera 10. The value “1” results for the area proportion 24c since this area portion of the patient 2 is aligned orthogonal to the camera 10. Due to the non-planar alignment relative to the camera 10, an area measurement of “66000” results for the total area 32.

In an alternative embodiment, the respective color values 20a-d are not determined directly from the values of the pixels 16a-d corresponding to the RGB values 22; rather, they are determined using a mask 38. The RGB values 22 are mapped by the mask 38 to two classes of color values A and B, wherein each color value hereby corresponds to a pathological property of the patient 2. The value A thus means that the corresponding classified pixel is such a pixel that depicts an inflamed region of the patient. A value B corresponds to a region of the patient that is not inflamed.

Therefore, not only individual RGB values 22 but respective value ranges in the RGB values 22 are also mapped to the values A and B using the mask 38. A standard color system 40 (for example the RAL color system) is used for this.

The figure schematically shows an additional embodiment in which respective groups of pixels in the endoscopy image 12 are assembled as a group 42, and a respective group 42 is depicted in an image point 18a-d and this is processed according to the cited method.

The figure shows with dashed lines a situation at a later point in time at which the endoscope 6 has been displaced further into the esophagus 4. Here additional regions 14 of the patient 2 are depicted in additional endoscopy images 12. The method described above can then be implemented at the individual endoscopy images 12 in order to generate an evaluation measure 28 for each endoscopy image 12.

Alternatively, the endoscopy images 12 can also be assembled into a sum image 44, or a 3D data set 46 can be reconstructed from these. The method described above can then be implemented on the sum image 44 or on the 3D data set 46, and an evaluation measure 28 can respectively be generated with regard to this.

Although modifications and changes may be suggested by those skilled in the art, it is the intention of the inventors to embody within the patent warranted heron all changes and modifications as reasonably and properly come within the scope of their contribution to the art.

We claim as our invention:

1-17. (canceled)

18. A method to process a digital endoscopy image of a patient comprising:
in a processor supplied with an endoscopy image comprised of a plurality of pixels, automatically identifying pixels, among said plurality of pixels, as image points that depict a body region of a patient;
in said processor determining a color value for each image point using a value of the pixel associated therewith;
in said processor, determining an area portion for each image point;
in said processor, determining, for each color value, a color area as a sum of all area portions of respective image points having the respective color value; and
in said processor, implementing an evaluation measure of said endoscopy image using the respective color areas.

19. A method as claimed in claim 18 comprising determining said area portion as a value correlated with an area of the pixel associated with the respective image point.

20. A method as claimed in claim 18 comprising determining said area portion as a value correlated with an area of the patient depicted by a respective image point.

21. A method as claimed in claim 18 comprising implementing said evaluation measure by determining a total area as a reference area for the respective color areas.

22. A method as claimed in claim 21 comprising determining said total area as an entirety of an image area of said endoscopy image.

23. A method as claimed in claim 21 comprising determining, as said total area, an area of the patient depicted in said endoscopy image.

24. A method as claimed in claim 21 comprising implementing said evaluation measure by calculating a quotient of a respect color area and said total area, for at least one of said color areas.

25. A method as claimed in claim 18 comprising implementing said evaluation measure by determining a histogram of said color areas over respective values of the color areas.

26. A method as claimed in claim 18 comprising selecting said color value to be correlated with a pathological property of the patient.

27. A method as claimed in claim 18 comprises employing value ranges of respective pixel values as said color values.

28. A method as claimed in claim 27 comprising using a standard color system to determine said value ranges.

29. A method as claimed in claim 18 comprising determining the respective values of said pixels as values of a plurality of color channels determined using a mask applied to said color channels.

30. A method as claimed in claim 18 comprising selecting the respective color values as respective luminosity-normalized 2D values.

31. A method as claimed in claim 18 comprising determining said image points as groups of pixels in said endoscopy image.

32. A method as claimed in claim 18 comprising implementing said evaluation measure for a plurality of different endoscopy images supplied to said processor.

33. A method as claimed in claim 18 comprising assembling said endoscopy image as a sum image of a plurality of individual endoscopy images.

34. A method as claimed in claim 18 comprising employing a 3D data set as said endoscopy image.
